(19) United States
(12) Patent Application Publication
(10) Pub. No.: US 2025/0263309 A1
(43) Pub. Date: Aug. 21, 2025

(54) GAS-TO-WATER ANALYZER FOR USE IN FLUID TREATMENT

(71) Applicant: SAUDI ARABIAN OIL COMPANY, Dhahran (SA)

(72) Inventor: Abdulaziz Yousef AL GHAREEB, Al Hofuf (SA)

(73) Assignee: SAUDI ARABIAN OIL COMPANY, Dhahran (SA)

(21) Appl. No.: 18/443,758

(22) Filed: Feb. 16, 2024

(51) Int. Cl. C02F 1/20; G01N 33/00

(52) U.S. Cl. CPC C02F 1/20; G01N 33/0073; C02F 2209/003; C02F 2209/40

(57) ABSTRACT

Examples are disclosed herein relating to fluid treatment. In an example, an analyzer that includes one or more first and second valves, a container, and a controller can receive a fluid. The first valve can provide the water to the container at a flow rate to fill up the container while the second valve removes the water from the container at a same or similar flow rate. A gas released from the fluid in the container during a period of time is collected. The controller receives measurements from sensors characterizing physical properties of the fluid and environment including a volume of the gas released from the fluid. The controller can determine an amount of the gas in the fluid based on the measurements. A flow rate and/or pressure of an inert gas that is being applied to the fluid can be adjusted based on the determined amount of the gas.

### FIELD OF THE DISCLOSURE

This disclosure relates generally to fluid treatment.

### BACKGROUND OF THE DISCLOSURE

Water treatment is a process of making water suitable for its intended use, whether for drinking, industrial applications, irrigation, river flow maintenance, or other purposes. This process involves removing contaminants and undesirable components from the water. Deaeration is a type of water treatment process used to remove dissolved gasses (e.g., oxygen) from water. and is used in industrial processes where the presence of dissolved gasses can lead to corrosion or interfere with industrial processes.

### SUMMARY OF THE DISCLOSURE

Various details of the present disclosure are hereinafter summarized to provide a basic understanding. This summary is not an extensive overview of the disclosure and is neither intended to identify certain elements of the disclosure nor to delineate the scope thereof. Rather, the primary purpose of this summary is to present some concepts of the disclosure in a simplified form prior to the more detailed description that is presented hereinafter.

According to an embodiment, a method can include introducing a fluid into a container, removing the fluid from the container, collecting gas released from the fluid in the container during a period of time, receiving measurements from sensors characterizing physical properties of the fluid and environment, determining an amount of the gas in the fluid based on the measurements, and causing a flow rate and/or pressure of an inert gas that is being applied to the fluid to be adjusted based on the determined amount of the gas. The received measurements can include a volume of gas released from the fluid in the container during the period of time.

According to another embodiment, a system can include an analyzer that can include a controller, a graduated eudiometer, one or more first valves that can be configured in response to the controller to provide water received from a pipeline to the graduated eudiometer to fill up the graduated eudiometer, one or more second valves that can be configured in response to the controller to remove the water from the graduated eudiometer, and sensors to measure physical properties of the water and an environment in which the analyzer is used to provide measurements during a period of time. The measurements can include a volume of gas released from the water in the graduated eudiometer during the period of time. The controller can be configured to determine an amount of the gas in the water as a gas to water (GTW) ratio based on the measurements.

In yet another embodiment, a system can include a deaeration system that can be configured to receive water at a water treatment facility and treat the water with an inert gas to remove one or more gasses from the water to output treated water into a pipeline for downstream processing. The system can further include a GTW analyzer that can be configured to determine an amount of one or more dissolved gasses in the treated water as GTW ratio. The GTW ratio can be used to cause the deaeration system to adjust a flow rate and/or pressure of the inert gas that is being used to treat the water in providing the treated water. The GTW analyzer can include a controller, a container, sensors, one or more first valves that can receive the treated water and provide the treated water to the container and one or more second valves that can remove the water from the container. The container can be configured to collect the one or more gas released from the treated water in the container for a period of time. The controller can be configured to receive measurements from the sensors characterizing physical properties of the treated water and environment during the period of time and determine the GTW ratio based on the measurements.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is an example of a water treatment system.

FIG. 2 is an example of a portion of a water treatment facility (or plant) implementing a deaeration process.

FIG. 3 is an example of a gas-to-water (GTW) analyzer.

FIG. 4 is an example of a table of variables for computing a GTW ratio according to one or more examples, as disclosed herein.

FIG. 5 is an example of a method of operating a GTW analyzer for computing GTW ratio in water (or water mixture).

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Water treatment plants, such as seawater plants (e.g., Qurayyah seawater plant), for example, are facilities of interconnected components that operate to treat water. Water treatment plants can be used in regions where water resources are scarce. One of the primary uses of seawater in the oil and gas industry is for Enhanced Oil Recovery (EOR) techniques. In EOR, water is injected into oil fields to help push the oil towards the extraction wells, increasing the amount of oil that can be recovered. Before seawater can be used for EOR, it must be treated to remove salts and other impurities that can damage the oil reservoirs or cause clogging in the injection wells. The desalination and purification processes at the seawater plant make seawater suitable for such use. The oil and gas industry often requires large amounts of cooling water for various processes, including refining and petrochemical production. Apart from EOR and cooling, the oil and gas industry can use water for various other purposes as well, such as steam generation, process water in refineries, and utility water for day-to-day operations.

To treat the water, the water is often drawn from a water source (e.g., a river, lake, ocean, etc.) using turbine pumps. These pumps are capable of moving large volumes of water into the treatment facility. As the water moves through pipelines, it can experience variations in flow rate and pressure. Surge tanks are installed within this pipeline network to absorb and mitigate these fluctuations, protecting the pipeline and pump systems. Water flows through various treatment stages (e.g., sedimentation, filtration, disinfection, etc.), facilitated by the pipeline system to get treated. Pumps may be used at different stages to maintain the required flow rate and pressure.

A water treatment process generally includes desalination (in some instances) to remove a salt content, which can cause scaling and other issues in reservoir, filtration and purification to remove particulate matter and other impurities, and deaeration to remove dissolved gasses to prevent corrosion and souring. A deaeration system is integrated into (or connected to) a pipeline infrastructure of the water treatment facility. The deaeration system is used to remove dissolved gasses, such as oxygen, from the water. The system achieves deoxygenation by injecting an inert gas, such as nitrogen gas into the water. When nitrogen gas is introduced into the water, it displaces one or more dissolved gasses, such as oxygen. The nitrogen gas is circulated through the water, causing the oxygen to be displaced and then removed from the water. However, if too much nitrogen gas is injected, it could lead to the formation of air bubbles or multi-phase flow. This is problematic in downward sloping pipes in the pipeline infrastructure where it can cause air pockets and potentially lead to a suction pressure drop.

Such pressure drops (suction pressure drops) can adversely affect equipment like gas turbine pumps or gravity pipes, which can lead to operational inefficiencies or damage. Gas turbine pumps in water treatment facilities (in the pipeline infrastructure) are used for moving large volumes of water for the treatment process. Gas turbine pumps are used to move water through the pipeline infrastructure, especially where a high pressure is needed to overcome resistance or to lift water to higher elevations. Gas turbine pumps can be used to draw water from the water source into the treatment facility (plant). These pumps, like any mechanical equipment, can be sensitive to operational conditions such as pressure drops or air entrainment, which can lead to inefficiencies or damage. Gravity pipes utilize a natural force of gravity to facilitate a flow of water, and do not require mechanical pumps for operation under certain conditions. Gravity pipes can be used in stages where water can flow naturally downhill or where the treatment process benefits from gravity-driven flow, such as in sedimentation tanks. An effectiveness of gravity pipes depends on the proper design of the system, including the slope and diameter of the pipes, to ensure adequate flow rates and prevent blockages.

In one or more examples herein, a gas to water (GTW) analyzer is disclosed that can be used to determine a GTW ratio (e.g., Nitrogen gas to water ratio) at atmospheric pressure and ambient temperature by a water displacement technique in a flowing system. The GTW analyzer can provide the GTW ratio, which can be used to control a rate and/or pressure of inert gas that is injected, for example, at a deaeration system. The GTW ratio can be used to control the rate and/or pressure of inert gas injected into the water that is being treated such that the amount of the inert gas that is injected results in a low likelihood of air bubbles or multi-phase flow forming. Thus, the GTW ratio can be used to control the rate and/or pressure of the inert gas such that suction pressure remains constant (or relatively constant (e.g., within a given amount or value of a design suction pressure), or from dropping, which can extend a life of mechanical equipment (e.g., turbine pumps, for example) used in water movement and/or treatment. In some examples, the GTW ratio can be used to control an air/vacuum valve (e.g., an APCO valve), an air release valve, and/or air relief valve and be used to identify an optimal location to install the air release valve or where the air pocket accumulates.

A flowing system as used herein refers to any system in which fluid (e.g., liquid or gas, or a combination of both) is in motion and can include a network of pipes, pumps, valves, and/or other components that facilitate a movement and/or treatment of one or more fluids. In a water treatment context, this can include pipelines (e.g., gravity pipes, etc.) carrying water, pumps (e.g., gas turbine pumps) driving the flow, treatment units (e.g., filters, sedimentation tanks, deaerators, etc.), valves, and control systems managing the flow. The pipeline infrastructure of the water treatment facility can be part of the flowing system, and can provide a physical pathway for water and/or other fluids to flow. Thus, in some examples, the flow system includes the deaeration system.

In some examples, the GTW analyzer can be configured to measure the GTW ratio in the flowing system to control and avoid a suction pressure drop or fluctuations in a pressure which can upset process conditions and cause inconsistent instrument sensing, as well as potentially compromise a quality of the water treatment. The data from the GTW analyzer can be used to control a deaeration process (the deaeration system). For example, the data can be to adjust the nitrogen gas that is injected based on real-time measurements so that operators can maintain optimal conditions, ensuring efficient removal of oxygen. Accordingly, the GTW analyzer can be used to improve an efficiency of water treatment surge tanks and pipelines in the treatment system. In some examples, the data from the GTW analyzer can be used to control the air/vacuum valve (e.g., an APCO valve), an air release valve, and/or air relief valve and to address downward sloping pipes or gravity pipe issues.

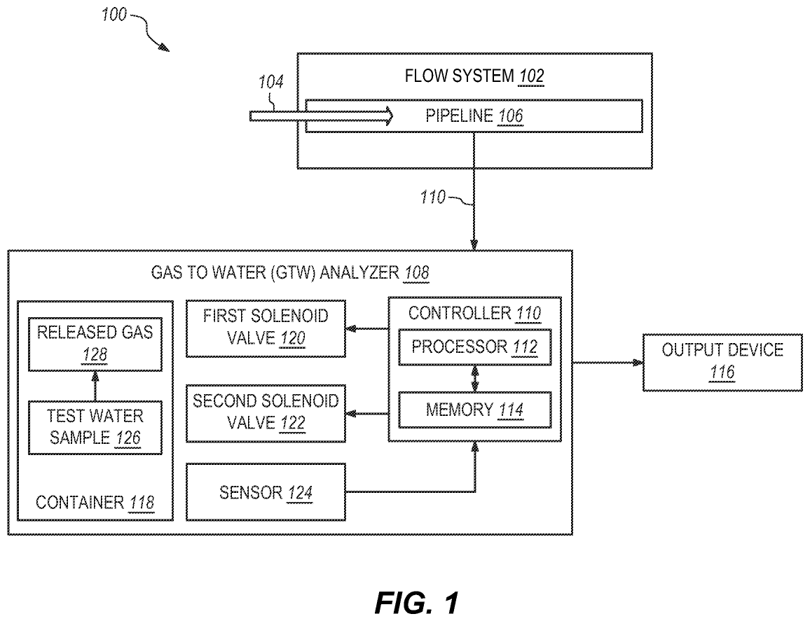

FIG. 1 is an example of a water treatment system 100 in which a GTW analyzer 108 is used. In some examples, the water treatment system 100 can be implemented as part of (or at) a water treatment plant or facility (e.g., Qurayyah seawater plant, in a non-limiting example). The water treatment system 100 includes a flow system 102. The flow system 102 can be used to facilitate a movement and/or treatment of water 104. In the examples herein, the water 104 is moved and/or treated by the flow system 102, but in other examples, a different fluid can be moved and/or treated, in some examples a multi-phase fluid. In some examples, the water 104 is provided as treated (or partially) treated water (e.g., saltwater) that has been extracted or provided from a source

In the example of FIG. 1, the water 104 is shown moving or flowing through a pipeline 106. In some examples, the pipeline 106 is part of a pipeline network, which can include or be used by or in connection with a deaeration system. The deaeration system can be integrated into (or connected to) a pipeline infrastructure of the water treatment facility. In other examples, the pipeline 106 is any pipeline of a pipeline network in which an air bubble or slug flow can form or occur. Thus, the pipeline 106 can be part of a system having slug or multi-phase flow. Slug flow is a flow regime characterized by intermittent passage of large gas bubbles (slugs) and liquid slugs through a pipeline. Multi-phase flow refers to a simultaneous flow of substances in different phases (solid, liquid, gas) within a pipeline or a process equipment. Unlike slug flow, the phases in multi-phase flow can be varied and dispersed. Multi-phase flow can exhibit various flow patterns, such as bubbly flow (small gas bubbles in liquid), stratified flow (distinct layers of gas and liquid), and annular flow (liquid along the walls with a gas core). Slug flow involves large intermittent gas pockets and liquid slugs, whereas multi-phase flow can involve any combination of solid, liquid, and gas phases in various forms and distributions.

A GTW analyzer 108 (or GTW analyzer system) can be used to compute a GTW ratio for the water 104 to determine an amount of (dissolved) gas (corresponding to gas 128) that is present in the water 104. For example, the GTW analyzer 108 can receive a portion of the water 104, which can be referred to as a test water sample 126. The GTW analyzer 108 can process the test water sample 126 to compute the GTW ratio. The GTW analyzer 108 can include a processor 112 and memory 114. The memory 114 can include machine readable instructions for computing the GTW ratio according to one or more examples herein and can be executed by the processor 112. The memory 114 can be any type of memory that can be accessed by the processor 112 to execute the machine readable instructions. The GTW analyzer 108 can be configured to determine the GTW ratio for the flowing system 102 to control and avoid a suction pressure drop or fluctuations in a pressure which can upset process conditions and cause inconsistent instrument sensing, and can potentially compromise a quality of the water treatment. The GTW analyzer 108 can provide the computed GTW ratio to an output device 116, as disclosed herein, in some examples.

For example, the GTW analyzer 108 can include a container 118, a first solenoid valve 120, a second solenoid valve 122 and sensors 124. In some examples, the container 118 is a cylinder. The controller 110 can cause the first solenoid valve 120 to provide the water 104 from the pipeline 106 at a flow rate to the container 118 to fill up the container 118 to a given level. The first solenoid valve 120 can be coupled to the container 118 and the pipeline 106. The controller 110 can cause the second solenoid valve 122 to remove the water 104 from the container 118 at a same flow rate as the first solenoid valve 120 provides the water 104 to the container 118 to fill up the container 118 so that the amount of the water in the container 118 remains at about the given level. The second solenoid valve 122 can be coupled to the container 118. The water in the container 118 can be referred to as the test water sample 126. The dissolved gas can be released from the test water sample 126 in the container 118. The released gas can be collected for a period of time to provide gas 128. The dissolved gas and the gas 128 can have a similar chemical nature. In a physical state, a dissolved state, gas is part of a liquid solution, and once released it exists independently in its gaseous form. Because of the similar chemical nature between the dissolved gas and the gas 128, the gas 128 can correspond to the dissolved gas, and can be referred to as dissolved gas. The sensors 124 can provide measurements characterizing physical properties of the water 104 and/or the test water sample 126, and an environment in which the GTW analyzer 108 is employed or used. The sensors 124 can include one or more sensors as disclosed herein from which data (signals) are used for computing or determining the GTW ratio. The measurements can include or characterize a volume of the gas 128 released from the water 104 and/or the test water sample 126 in the container 118 during the period of time. The controller 110 can determine an amount of gas in the water as the GTW ratio based on the measurements.

The GTW analyzer 108 can be used to measure a ratio in different forms of gas to liquid ratio or nitrogen gas to water ratio in the pipeline 106 or the flowing system 102, which could contain (air bubble) two phase or gas bubbles and liquid in the flowing system 102. The GTW analyzer 108 can be used in a field of water treatment, such as in deaeration systems which use an inert gas (e.g., nitrogen gas) to remove dissolved oxygen gas or air in the water 104. In other examples, the GTW analyzer 108 can be used to serve the slug flow and multiphase flow in any piping network. The GTW analyzer 108 can provide continuous readings and results in a form of rates, percentages and/or cubic feet per barrel for the engineers and the operation team. The GTW analyzer 108 could be used to assist engineers and the operation team to design a pipeline network and selecting a proper place to install an an air/vacuum valve (e.g., an APCO valve) and/or a proper size for the air/vacuum valve, an air release valve, and/or air relief valve, which can be used to remove an air pocket to help control an operation and prevent suction pressure drop or pressure fluctuations at a gas turbine pump.

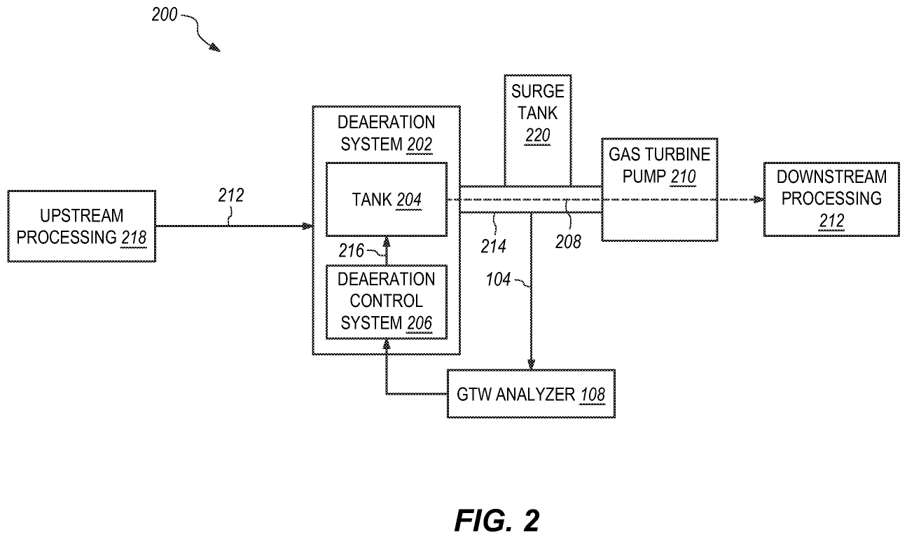

FIG. 2 is an example of a portion of a water treatment facility (or plant) 200 implementing a deaeration process. The plant 200 can be configured to receive water 212, which can be provided from upstream processing 218, as shown in FIG. 2. Thus, reference can be made to one or more examples of FIG. 1 in the example of FIG. 2. The plant 200 includes a deaeration system 202 for deaeration of the water 212. The water 212 can be received by the deaeration system 202. For clarity and brevity purposes, the deaeration system 202 is a simplified example of a deaeration system that can be used to treat the water 212. The deaeration system 202 includes a tank 204 that can be filled with the water 212. The tank 204 is first filled with the water 212 that needs to be treated. This water 212 can include dissolved gasses, including oxygen, which need to be removed. Once the water is in the tank 204, inert gas 216 (e.g., nitrogen gas) can be injected into the tank 204. The method of injection can vary; it can involve sparging, where gas is bubbled through the water 212 from the bottom of the tank 204, or it might involve a spray-type system where the water 212 in the tank 204 is sprayed through a gas-filled column.

A deaeration control system 206 can be used to control how the inert gas 216 is injected into the tank 204. Nitrogen, being less soluble in the water 104 than oxygen, can help remove the oxygen by forcing it out of solution. The oxygen can then either rise to the top of the tank 204 or can be otherwise vented out of the deaeration system 202. The deaeration control system 206 can control and monitor a deaeration process at the deaeration system 202. The deaeration control system 206 can regulate a flow rate and/or pressure of the inert gas 216 that is injected, in some examples, as well as continuously monitoring the oxygen levels in the water 104 in the tank 204 to ensure effective deaeration. In some examples, the GTW analyzer 108 can communicate with the deaeration control system 206 to receive the GTW ratio and use the GTW ratio to adjust the flow rate and/or pressure of the inert gas 216 that is injected into the tank 204. For example, the GTW analyzer 108 can be configured to receive treated water 208 provided by the deaeration system 202 and compute the GTW ratio using the treated water 208 according to one or more examples as disclosed herein. In some examples, the GTW ratio can be used to adjust the flow rate and/or the pressure of the inert gas 216 that is injected into the tank 204. In some examples, a different system or device can be used to process the GTW ratio to determine a new flow rate and/or pressure for injecting the inert gas 216 into the tank 204. In another example, the GWR analyzer 108 can be utilized for venting and removing gases to or from the air/vacuum valve, such as an APCO valve. This includes use with an air release valve, an air relief valve, or any other device monitoring the ventilation of the tank. It can also be applied to any device in the system, including pipes and tanks, that requires monitoring for gas presence or pressure regulation. The density of water 104 before flowing through the container 118 and during the test can change. Since the density could vary during the test due to a temperature change this can affect an amount of dissolved gas.

After deaeration, the water 212 in the tank 204 is typically free from dissolved oxygen and can be provided as treated water 208. The treated water 208 can then be conveyed (transported), either by gas turbine pumps or gravity flow, depending on system design. The example of FIG. 2 illustrates conveyance of the treated water 208 using a gas turbine pump 210 downstream in the plant 200 for further processing, referred to as downstream processing 212 in the example of FIG. 2. For example, the treated water 208 can be released from the tank 204 to the gas turbine pump 210 using a pipeline (or pipeline network) 214. In some examples, the treated water 208 is the water 104, as shown in FIG. 1. The pipeline 214 can include one or more downward sloping pipes. A surge tank 220 can be coupled to the pipeline or pipeline network 214 to control pressure changes due to changes in water flow rate of the treated water 208.

Accordingly, by including the GTW analyzer 108 in the plant 200, a production of the plant 200 can be increased (e.g., by a given percentage relative to a prior plant production capability), and can improve improving an efficiency of water treatment surge tanks (e.g., the surge tank 220) and pipelines network (e.g., the pipeline or pipeline network 214, and/or other pipeline networks (or pipeline)) downstream that are used at downstream processing 212. Furthermore, a lifespan of the gas turbine pump 210 is extended through the use of the GTW analyzer 108 in the plant by detecting pressure drops in the pipeline 214, which could cause the gas turbine pump 210 to trip. For example, the GTW ratio can be compared to at GTW ratio threshold (value) that is correlated with (or associated) with a pressure drop that if experienced by the gas turbine pump 210 would cause the gas turbine pump 210 to trip, which can result in damage to the gas turbine pump 212. In some examples, the controller 110 can be used to compare the GTW ratio to the GTW ratio threshold and if the GTW ratio is greater than or equal to the GTW ratio threshold this can indicate that is a high likelihood that the gas turbine pump 210 will trip because of the pressure drop caused by the injected inert gas (e.g., when to much nitrogen gas injected for treatment of the water 212). In some instances, operators can be notified before the pressure drop occurs by the GTW analyzer 108. For example, if there is a high likelihood that the gas turbine pump 210 will trip, the controller 110 can issue a notification, which can be provided to an operators device (e.g., user device as disclosed herein) wirelessly and/or over a wired connection as an alert (e.g., an email, a message, audible sound, etc.) In some examples, the notification can be provided to a distributed control system (DCS) or Programmable Logic Controller (PLC). Furthermore, the GTW analyzer 108 prevents cavitation to ensure optimal operation of the gas turbine pump 210, which can reduce maintenance cost (e.g., in some instances by about 17%), maximize water production and ensure a consistent flow of treated water 208 to the gas turbine pumps 210 by minimizing a risk of bubble formation (e.g., in some instances by about 90%).

Accordingly, the use of the GTW analyzer 108 can be used to solve a suction pressure drop problem that can be experienced by the gas turbine pump 210 when to much of the inert gas 216 is used for treatment. The GTW analyzer 108 can be configured to give continuous data to operation and/or engineers, regarding a GTW ratio which can be related directly to the suction pressure drop. By using the GTW ratio results, the suction pressure drop can be estimated, which can help avoid any tripping to the gas turbine pump 210. In some instances, the gas turbine pump 210 can be located at a downstream plant relative to a water treatment plant. In some instances, using or installing the GTW analyzer 108 at different samples points and connected to a board (e.g., of a DCS or PLC system) can assist engineers in selecting a proper place to install an air release valve or APCO valve, and take advance action such to avoid the suction pressure drop. The sampling points location can be provided by a process engineer, in some instances.

Because the GTW results can change over time depending on a nitrogen dosage, the GTW analyzer 108 can be used to provide a continuous reading using a closed loop system, and use a PID controller to assure an accuracy of measurements, for example, as described herein. In some examples, a custom operation mode of the GTW analyzer 108 can be used to ensure continuous readings (and thus continuous data). The GTW analyzer 108 can estimate a volume of bubble flow regardless of what a flow and pressure is. The GTW analyzer 108 can be implemented in some instances as a portable analyzer and/or can be coupled to a sampling point. Thus, in some examples, the GTW analyzer 108 can be implemented as a standalone device or instrument. The device is capable of working by itself and can share continuous data to the engineers. The GTW analyzer 108 can be adapted to be suitable for various industries, including water treatment plants, such as the water treatment plant 200, as shown in FIG. 2. Thus, the GTW analyzer 108 can be used as part of processing monitoring operations.

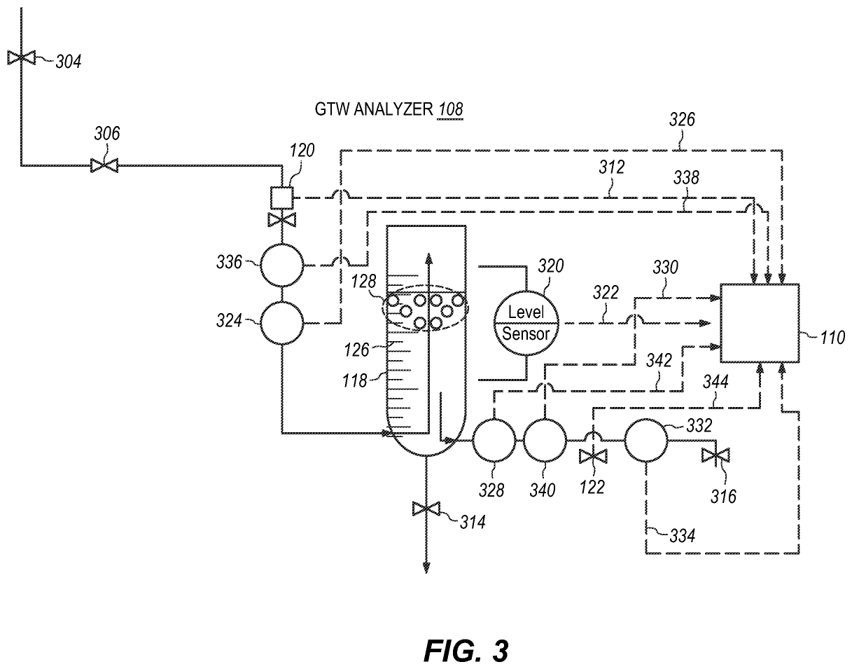

FIG. 3 is an example of the GTW analyzer 108 according to one or more examples as disclosed herein. Thus, reference can be made to one or more examples of FIGS. 1-2 in the example of FIG. 3. The GTW analyzer 108 can receive the water 104 (or the water 208). For example, the GTW analyzer 108 can be coupled to a manual valve 304, which in some instances, can be referred to as a sampling point. Once coupled to the manual valve 304, the manual valve can be engaged (e.g., by a user) so that a portion of the water 104 flows to the GTW analyzer 108. The GTW analyzer 108 includes a gate valve 306. The gate valve 306 can be implemented as an electromechanical gate valve, which can be initiated (e.g., to a fully open or fully closed position by a user, for example). The gate valve 306 can be used to flush a container 118, which is shown in the example of FIG. 3 as a cylinder. The gate valve 306 can be used before or during a gas volume test to flush the container 118. The gas volume test refers to a procedure for collecting a volume of gas from a sample of the water 104 (the test water sample 126, as shown in FIG. 3). The container 118 can be a graduated eudiometer. The gate valve 306 can be placed in a fully open position from a fully closed position to provide the water 104 to the container 118.

In some examples, the GTW analyzer 108 includes the first solenoid valve 120. The first solenoid valve 120 in some instances can be implemented as an electromechanical valve that can be actuated or activated to control an inlet flow of the water 104 to the container 118. For example, the first solenoid valve 120 can receive an inlet control signal 312 from the controller 110 that can cause the first solenoid valve 310 to allow the water 104 to flow to the container 118 from the gate valve 306. In some examples, a proportional-integral-derivative (PID) controller can be used to control the flow of the water 104 to the container 118. The controller 110 can be implemented using one or more modules. The one or more modules can be in software and/or hardware form, or a combination thereof. In some examples, the controller 110 can be implemented as machine-readable instructions for execution on a computing platform (or system). Thus in examples in which the controller 110 is implemented as machine-readable instructions, the controller 110 can be executed by a central processing unit (CPU) of a computing platform.

The water 104 can be allowed to flow into the container 118 for the gas volume test. The container 118 can collect the water 104, and can have an inlet tube, an outlet tube and drain tube. The inlet tube can be used to receive the e water 104, the outlet tube can be used to provide the water 104 to a second drain valve 316, and the drain tube can be used to drain the portion of the water 104 from the container 118. In some examples, the second drain valve 316 is a manual drain valve. In some examples, a first drain valve 314 can be used to drain the container 118. For example, the first drain valve 314 can be used to drain the container 118 before the gas volume test. The first drain valve 314 can be implemented as an electromechanical gate valve. The first drain valve 314 can be placed from a fully closed position to a fully open position to receive the water 104 from the container 118 via the drain tube to remove the water from the container 118.

In some examples, a second solenoid valve 122 can be used to control an outlet flow of the water 104 in the container 118 to the second drain valve 316, as shown in FIG. 3. For example, the second solenoid valve 122 can receive an outlet control signal 344 from the controller 110 that can cause the second solenoid valve 122 to allow the water 104 to flow from the container 118 to the second drain valve 316. In some examples, the second solenoid valve 122 is implemented as an electromechanical valve. In some examples, a PID controller can be used to control the flow of the water 104 to the second drain valve 316. A level sensor 320 can be used to monitor (or measure) a level or an amount of gas (collected gas) V_{g} in the container 118 (e.g., during testing). In some examples, the level sensor 320 is a eudiometer level sensor. The level sensor 320 can be implemented as an ultrasonic sensor in some instances so that the container 118 remains a closed-loop system to prevent any likelihood (or chance) that air affects a reading (or measurement), as disclosed herein. The level sensor 320 can provide a level sensor signal 322 indicative of the amount of gas (volume of gas) in the container 118.

The GTW analyzer 108 can further include a first temperature sensor 324 to measure (read) an ambient temperature T of (ambient) air in an environment in which the GTW analyzer 108 is used, and provide a first temperature signal 326 indicative of the temperature of the air. A second temperature sensor 328 can be used in the GTW analyzer 108 to measure (read) a temperature of the water 104 (e.g., in the container 118 or flowing through the container 118) and provide a second temperature signal 330 indicative of the temperature of the water 104 (e.g., an instance in time or over a period of time). The controller 110 can measure the temperature of the water 104 (the test water sample 126) over a period of time that was used during the gas volume test (e.g., in the container 118) and compute an average water temperature TAVG based on the second temperature signal. The GTW analyzer 108 can further include a water recorder device 332 (also known as a water outlet recorder in some instances). The water recorder device 332 can be used to record (measure) one or more parameters of the water 104. In some examples, the water recorder device 332 is a flowmeter (or flow sensor) and measures a volume of the water V_{w} 104 that flows through the second solenoid valve 122 and provide a water volume signal 334 indicative of the volume of water V_{w} 104 (e.g., during the test). The first solenoid valve 120 and second solenoid valve 122 can be adjusted at a beginning of the gas volume test or prior so that a flow rate of an inlet water (the water 104 flowing to the container 118) and a flow rate of an outlet water (the water 104 from from the container 118). The PID controller (or each PID controller) can be used to adjust the flow rate of the inlet and outlet water so the flow rate is steady during the gas volume test. For example, the flow rate can be set to about 10 to about 12 liters/hour.

The GTW analyzer 108 can further include a first electrical conductivity meter 336 that can be used to measure an electrical conductivity of the water 104 that is being provided to the container 118. The first electrical conductivity meter 336 can output a first electrical conductivity signal 338 indicative of the electrical conductivity of the water 104 being used to fill the container 118. The GTW analyzer 108 can also include a second electrical conductivity meter 340 that can be used to measure an electrical conductivity of the water 104 after flowing from the container 118. The second electrical conductivity meter 340 can output a second electrical conductivity signal 342 indicative of the electrical conductivity of the water 104 being provided by (or from) the container 118. The controller 110 can receive the first and second electrical conductivity signals 338-340 and estimate a total dissolved solid (TDS) content of the water 104. In some examples, each of the first and second electrical conductivity meters 338-340 can include a built-in conversion factor to estimate the TDS from conductivity measurements. The TDS can be related to a density of the water 104, as shown in expression (1). The TDS can be used to estimate a density of the water 104. Temperature is an important factor in the density of liquid so it can provide accurate readings regarding the TDS.

For example, to determine the GTW ratio for the water 104 (the test water sample 126), the GTW analyzer 108 can be configured to collect the gas from the water 104 by using one of a laboratory techniques to collect the gas referred to as water displacement (e.g., trap in water). The water 104 can flow through an inverted graduated eudiometer (the container 118), flashed out gasses (e.g., oxygen gas that is released from the water 104) can be collected in the container 118 while a water volume of a throughput (e.g., the volume of the water V_{w} 104) is measured by a flowmeter. Because the container 118 is graduated, it allows for a measurement of a volume of gas V_{G} collected. The water 104 can be allowed to flow through the container 118 for a period of time (e.g., from about 45 minutes to about 60 minutes) and the gasses from the water 104 can be trapped in the container 118. The period of time can be referred to as a gas trapping period as gasses are captured during this time frame. After the gas trapping period, or at an end of the gas trapping period, the first solenoid valve 120 and second solenoid valve 122 can be deactivated or turned off and an amount of the water in the container 118 can be measured. An amount of water in the container 118 can be measured at an outset of the gas trapping period (e.g., when an inlet and outlet flow rate are about the same) or at about a beginning of the gas trapping period, and after or toward the end of the gas trapping period. For example, a liquid level sensor (e.g., within the container 118) can be positioned and used to measure an initial amount of the water 104 (the test water sample 126) in the container 118 at the outset of the gas trapping period or at about the beginning of the gas trapping period. The liquid level sensor can be used to measure a final amount of the water 104 (the test water sample 126) in the container 118 after or toward the end of the gas trapping period. The controller 110 can subtract the initial amount of the water 104 from the final amount of the water 104 in the container 118 to determine the volume of gas V_{G} collected. During the gas trapping period, the pressure must be equal or close to an atmospheric pressure by keeping a level of the outlet tube of the container 118 even with a water level in the pipeline 106 or the pipeline 214, which is applied to the sampling point too. The pressure measurement can be optional. During the installation of the GTW analyzer 108 at the sampling point, this pressure can be estimated. In some examples, to guarantee accurate readings, the user can manually input this estimated pressure value and the estimated pressure can be provided to the controller 110. The pressure can be provided by using a pressure measurement system and/or the user can manually input this estimated pressure value and the pressure can be provided to the controller 110. However, a pressure regulator can be added as a feature before the water 104 enters the analyzer 108 to avoid any pressure changes that could affect the analyzer readings and to ensure efficiency.

In some examples, during the gas volume test, a TDS, a specific gravity, a temperature and an ambient pressure can be recorded or captured during the gas volume test (during the period of time) for computing the GTW ratio. The temperature is an average temperature during the test based on a test length since the container 118 temperature could vary depending on atmospheric temperature. The controller 110 can receive data or signals indicative of the TDS, the specific gravity, the temperature and the pressure. TDS refers to a total concentration of dissolved substances in the water 104, for example, minerals, salts and/or metals. The amount of gas that can dissolve in the water 104 can be affected by a concentration of the dissolved substances. Generally, water with a higher TDS can have a lower capacity to dissolve additional gasses. Specific gravity refers to a ratio of density of a substance to a density of a reference substance (typically water). Specific gravity can be used to provide information about a composition of the water 104.

The controller 110 can compute (or obtain) the GTW ratio (e.g., Nitrogen gas to water ratio, for example). The GTW ratio can be represented as a percentage, or can be converted by the controller 110 to cubic feet per barrel. The GTW ratio can be found using the SI unit and American unit. For example, the controller 110 can compute the GTW ratio according to the following expression (formula):

$$GTW = \frac{( V g ) ( T ) ( P - p ) ( D o )}{( V w ) ( T a v g ) ( P a ) ( D t )} . \qquad ( 1 )$$

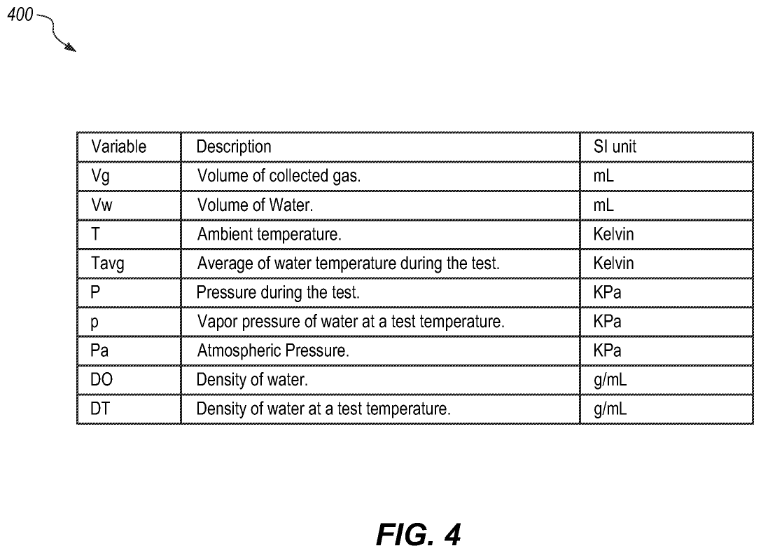

FIG. 4 is an example of a table 400 of variables for the expression (1). The table 400 identifies each variable of the expression (1) and provides a brief explanation of the variables represent (or mean) and an associated unit for each variable. The pressure variables P, p, and Pa in expression (1) can be provided by a user or another system, while the density of water DO and density of water at a test temperature DT can be provided based on the TDS provided by one of the first and second electrical conductivity meters 336-338 (or computed by the controller 110). For example, the first electrical conductivity meter 336 can be used to provide the density of the water DO 104 and the second electrical conductivity meter 336 can be used to provide the density of the water 104 at the test temperature DT. For example, the pressure can be provided by using a pressure measurement system and/or the user can manually input this estimated pressure value and the pressure can be provided to the controller 110. However, a pressure regulator can be added as a feature before the water 104 enters the analyzer 108 to avoid any pressure changes that could affect the analyzer readings and to ensure efficiency.

For example, in a given example, if the gas collected in the container 118 is about 250 millimeters (mL) and a volume of the water is 8000 mL, using the expression (1), the GTW ratio is 0.02288 or 2.29%.

In some examples, to define the GTW ratio as a ratio of gas to water per barrel (GTW_{B}) ratio, the following expression (formula) can be used by the controller 110:

$$G T W \times 5.615 = G T {W}_{B} , \qquad ( 2 )$$

wherein 5.615 cubic feet is about one (1) barrel.

In the given example, the GTW_{B} ratio can be 0.03215 cubic feet per barrel

In some examples, the controller 110 can operate in two operating modes (a first operating mode and a second operating mode) based on which variables (or information) is available to the controller 110 for computing the GTW ratio. In the first mode, all data (or values for the variables) in the expression (1) are available, and the controller 110 can compute the GTW ratio according to one or more examples as disclosed herein. The second mode can be used when not all of the data is available for computing the GTW ratio. In some examples, the second mode is a custom mode. The second mode can be selected by a user. The user can provide user input to the controller 110 or a command/instructions that can identify which variables from the expression (1) that the controller 110 should not consider in computing the GTW ratio. The second mode allows for the GTW ratio to be calculated in scenarios in which sensors have been damaged or providing measurements with errors. The sensors can include one or more sensors, as disclosed herein. The second mode can be used by the user to avoid manipulation in the results as results of the damage. The second mode (feature) of the controller 110 allows for the GTW ratio to be computed while a damaged sensor is fixed or maintained and brought back online.

Accordingly, the water 104 can be introduced into the container 118 through the first solenoid valve 120 at a flow rate, which can be referred to as a first flow rate. The water 104 can be removed from the container 118 using the second solenoid valve 122 so that the amount of the water 104 in the container 118 remains substantially the same. In some examples, the controller 110 or another device or system can determine the first flow rate of the water 104 into the container 114 and a second flow rate of the water 104 from the container 118. The controller 110 (or another system or device) can adjust one of the first and second flow rates so that the amount of the water 104 in the container 118. remains substantially the same. The gas released from the water 104 can be collected in the container 118 during a period of time. The controller 110 can receive measurements from one or more sensors as disclosed herein that can characterize physical properties of the water 104 and/or environment. The received measurements can include a volume of gas released from the water 104 in the container 118 during the period of time. The measurements can further include a volume of the water 104 that has been removed from the container 118 during the period of time. In some examples, the measurements can further include a temperature of the water 104 during the period of time. In some examples, an average temperature for the water 104 can be used for computing an average temperature of the water 104, which can be used for determining the amount of gas in the water 104. In some examples, the measurements can include an ambient air temperature, a pressure of the water 104 in the container 118, a vapor pressure of the water 104 in the container 118, and an atmospheric pressure during the period of time. The measurements in some examples can include a density of the water 104 before the period of time, and a density of the water 104 after the period of time. An amount of the gas in the water 104 can be determined based on the measurements. Since the density could vary during the test due to a temperature change this can affect the dissolved gas. In some examples, a flow rate and/or pressure of an inert gas that is being applied to the water 104 can be adjusted based on the determined amount of the gas.

In some instances, a system may comprise a treatment setup that includes a deaeration system and a surge tank, along with an analyzer as disclosed herein equipped with additional valves, a graduated eudiometer, and sensors. These components can be designed to calculate the GTW ratio, which can be used for controlling devices, such as air/vacuum valves (for example, an APCO valve), air release valves, and air relief valves. These valves can be used to eliminate air pockets, thereby ensuring the smooth operation of downstream processes.

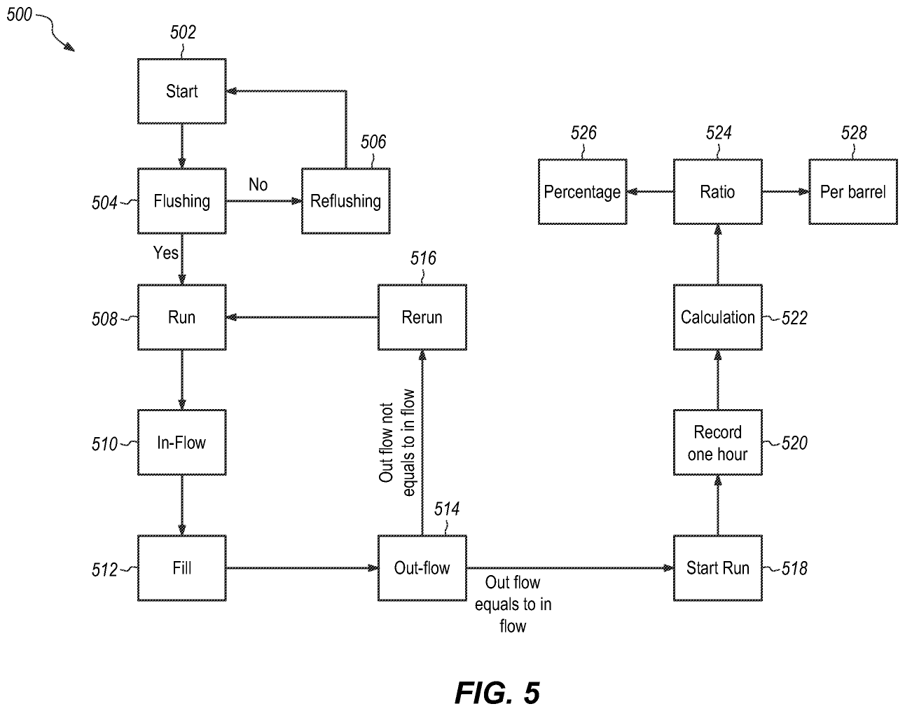

In view of the foregoing structural and functional features described above, an example method will be better appreciated with reference to FIG. 5. While, for purposes of simplicity of explanation, the example method of FIG. 5 is shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the method.

FIG. 5 is an example of a method 500 of operating the GTW analyzer 108 to compute a GTW ratio for the water 104, as shown in FIG. 1, or the water 208, as shown in FIG. 2. Thus, reference can be made to one or more examples of FIGS. 1-4 in the example of FIG. 5. At 502 the method 500 can begin. At 504, the container 118 can be flushed. In some examples, the container 118 can be flushed before a gas volume test, or as part of the gas volume test. For example, the water 104 can be provided to the container 118 to fill up the container 118 (the graduated eudiometer). The water 104 in the pipeline 106 can be provided to the manual valve 304, which can be opened (e.g., by an engineer) so that the water 104 flows to the gate valve 306. The gate valve 306 can be placed in an open position (e.g., in some instances by the controller 110, as shown in FIGS. 1 and 3) so the water 104 flows to the first solenoid valve 310. The first solenoid valve 120 can be activated or actuated (e.g., by the controller 110 in some instances) and the water 104 can fill the container 118 while the first drain valve 314 (outlet drain valve) and the second solenoid valve 122 are closed (e.g., in a closed state).

After the container 118 is filled up, the gate valve 306 can be closed and the first drain valve 314 can be opened (e.g., by the controller 110 in some instances) and the water 104 in the container 118 can start draining to ensure no residual air or water is in the container 118 for subsequent steps of the gas volume test. In some examples, the method 500 can proceed to step 506 (shown as “No” in FIG. 5) in response to determining that the container 118 needs to be re-flushed (e.g., when residual air or water resides in the container 118), and steps 502 and 504 can be repeated. If no re-flushing is needed, the method 500 can proceed to step 508 at which the gas volume test can begin.

At 508, the first solenoid valve 120 can be controlled so that an in-flow rate of the water 104 into the container 118 and an outflow-rate of the water 104 from the container 118 is about the same. For example, the water 104 can be provided from the pipeline 106 through the manual valve 304 (while in an open position) to the gate valve 306. The gate valve 306 can be controlled (e.g., in some instances by the controller 110) to provide the water 104 to the first solenoid valve 310. At 510, the first solenoid valve 120 can be controlled (e.g., in some instances by the controller 110) to control the in-flow rate of the water 104 into the container 118 to fill up the container 118 at 512 to a given level. In a non-limiting example, the flow rate is about 170 to about 200 mL/sec.

At 514, the second solenoid valve 122 can be controlled (e.g., in some instances by the controller 110) so that out-flow rate of the water from the cylinder can be controlled. A flow rate of the water (the test water sample 126) in the container 118 that is being removed by the second solenoid valve 122 can be about the flow rate at which the water is provided by the first solenoid valve 120 to the cylinder 318. Thus, an even flow rate can be established between an inlet and outlet of the cylinder 318 so that an amount of the water 104 in the container 118 is kept constant (e.g., at a given level or within a particular or desired percentage (e.g., within about 0 to about 10%, or some other percentage), or value. For example, at 514, a determination can be made (e.g., in some instances by the controller 110) to determine whether the in-flow rate is about the same as the out-flow rate. If the in-flow rate is not about the same as the out-flow rate (shown as “Out flow not equals to the in flow” in FIG. 5), the method 500 can proceed to step 516, at which steps 508-514 can be repeated (rerun) until the determination results in the in-flow rate and the out-flow rate being about the same. The method 500 can then proceed to step 518 (shown as “Out flow equals to in flow” in FIG. 5).

At 518, measurements from one or more sensors as disclosed herein can be received (e.g., by the controller 110) for computing the GTW ratio, which is referred to as “Start Run” in the example of FIG. 5. At 518, the measurements from the one or more sensors can be received over a period of time (e.g., a gas trapping period). In some examples, at 518, the amount of gas in the container 118 can measured or received (e.g., by the controller 110, as shown in FIG. 1), such as using the level sensor 320 and the water recorder device 332 (e.g., flowmeter) can be used to measure how much water goes through the second solenoid valve 122. In the example of FIG. 5, the period of time is one (1) hour but other time periods are contemplated within the scope of this disclosure. At 520, the GTW ratio can be calculated (e.g., by the controller 110) as disclosed herein. In some examples, at 520 the measurements: gas volume inside the eudiometer, water outlet volume (e.g., about 8000 to about 12000 L), temperature, pressure and TDS can be used to calculate the GTW ratio. In some examples, before or after step 520, the first drain valve 314 can be opened to drain the remaining water from the container 118. At 524, the GTW ratio can be provided to the output device 116 (e.g., rendered on a display for a user). In some examples, the output device 116 is a user device. Example user devices can include, for example, a mobile phone, a portable device, a tablet, a desktop computer, a laptop computer, or any other type of device that the user can use and retrieve the GTW ratio. In some examples, at 526, the GTW ratio can be represented as a percentage, and in some instances provided to the output device 116. In some examples, at 528, the GTW ratio can be represented as per barrel, and in some instances provided to the output device 116.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a standalone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts or carry out combinations of special purpose hardware and computer instructions.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof. In addition, the use of ordinal numbers (e.g., first, second, third, etc.) is for distinction and not counting. For example, the use of “third” does not imply there must be a corresponding “first” or “second.” Also, as used herein, the terms “coupled” or “coupled to” or “connected” or “connected to” or “attached” or “attached to” may indicate establishing either a direct or indirect connection, and is not limited to either unless expressly referenced as such. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The term “based on” means “based at least in part on.” The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 5-10% of the indicated number.

What has been described above include mere examples of systems, computer program products and computer-implemented methods. It is, of course, not possible to describe every conceivable combination of components, products and/or computer-implemented methods for purposes of describing this disclosure, but one of ordinary skill in the art can recognize that many further combinations and permutations of this disclosure are possible. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

The invention claimed is:

1. A method comprising:
introducing a fluid into a container;
removing the fluid from the container;
collecting gas released from the fluid in the container during a period of time;
receiving measurements from sensors characterizing physical properties of the fluid and environment, the received measurements including a volume of gas released from the fluid in the container during the period of time;
determining an amount of the gas in the fluid based on the measurements; and
causing a flow rate and/or pressure of an inert gas that is being applied to the fluid to be adjusted based on the determined amount of the gas.

2. The method of claim 1, wherein the measurements further includes a volume of the fluid that was removed from the container during the period of time.

3. The method of claim 1, wherein the measurements further include a temperature of the fluid during the period of time, and the method further comprising computing an average temperature for the fluid, the average temperature of the fluid being used for determining the amount of gas in the fluid.

4. The method of claim 3, wherein the measurements further include an ambient air temperature, a pressure of the fluid in the container, a vapor pressure of the fluid in the container, and an atmospheric pressure during the period of time.

5. The method of claim 4, wherein the measurements further include a density of the fluid before the period of time, and a density of the fluid after the period of time.

6. The method of claim 1, wherein the amount of gas in the fluid is determined as a gas to water (GTW) ratio.

7. The method of claim 1, wherein the GTW ratio is represented as a percentage.

8. The method of claim 1, wherein the GTW ratio is represented as cubic feet of gas per barrel.

9. The method of claim 1, further comprising:
adjusting a rate at which fluid is provided and removed from the container so that an amount of fluid in the container remains substantially the same.

10. The method of claim 1, wherein the causing, collecting, receiving and determining steps are performed after flushing the container to remove residual air or fluid in the container.

11. The method of claim 10, wherein the flushing comprises:
causing a one or more first valves to receive the fluid from an upstream valve so that the fluid flows to the container; and
causing one or more second valves to provide the fluid to the container to fill up the container.

12. The method of claim 11, wherein the flushing further comprises:
setting the one or more second valves to a closed state to stop the fluid from being provided to the container; and
setting one or more third valves to an open state to drain the fluid in the container to remove the residual air or water in the container.

13. The method of claim 10, repeating the flushing for a number of steps until the residual air or fluid is removed from the container.

14. The method of claim 1, wherein the container is a graduated eudiometer.

15. A system comprising:
an analyzer comprising:
a controller;
a graduated eudiometer;
one or more first valves configured in response to the controller to provide water received from a pipeline to the graduated eudiometer to fill up the graduated eudiometer;
one or more second valves configured in response to the controller to remove the water from the graduated eudiometer;
sensors to measure physical properties of the water and an environment in which the analyzer is used to provide measurements during a period of time, the measurements including a volume of gas released from the water in the graduated eudiometer during the period of time; and
wherein the controller is configured to determine an amount of the gas in the water as a gas to water (GTW) ratio based on the measurements.

16. The system of claim 15, wherein the controller is further configured to:
determine a first flow rate of the water into the cylinder and a second flow rate of the water from the cylinder; and
adjust one of the first and second flow rates so that an amount of water in the cylinder remains about a same during the period of time.

17. The system of claim 15, wherein the measurements further include a temperature of the water, an ambient air temperature, a pressure of the water in the cylinder, a vapor pressure of the water in the container, and an atmospheric pressure during the period of time.

18. The system of claim 17, wherein the measurements further include a density of the water before the period of time, and a density of the water after the period of time.

19. A system comprising:
a deaeration system configured to receive water at a water treatment facility and treat the water with an inert gas to remove one or more gasses from the water to output treated water into a pipeline for downstream processing;
a gas to water (GTW) analyzer configured to determine an amount of one or more dissolved gasses in the treated water as a GTW ratio, the GTW ratio being used to cause the deaeration system to adjust a flow rate and/or pressure of the inert gas that is being used to treat the water in providing the treated water;
wherein the GTW analyzer comprises:
a controller;
a container;
sensors;
one or more first valves to receive the treated water and provide the treated water to the container;
one or more second valves to remove the water from the container, wherein:
the container is configured to collect the one or more gasses released from the treated water in the container for a period of time; and
the controller is configured to receive measurements from the sensors characterizing physical properties of the treated water and environment during the period of time and determine the GTW ratio based on the measurements.

20. The system of claim 19, wherein the GTW ratio is used to control an air/vacuum valve, an air release valve, and/or air relief valve, and/or another system or device to control a downstream process.
